(19) United States
(12) Patent Application Publication
(10) Pub. No.: US 2007/0123877 A1
(43) Pub. Date: May 31, 2007

(54) Inflatable Device for Restoring Anatomy of Fractured Bone

(75) Inventors: Mark Goldin, Orlando, FL (US); Brian Schumacher, Orlando, FL (US); William Christy, Winter Park, FL (US); Melvin Rosenwasser, Palisades, NY (US)

Correspondence Address: FROST BROWN TODD, LLC, 2200 PNC CENTER, 201 E. FIFTH STREET, CINCINNATI, OH, 45202, US

(73) Assignee: AOI Medical, Inc., Orlando, FL (US)

(21) Appl. No.: 11/559,993

(22) Filed: Nov. 15, 2006

Related U.S. Application Data
(60) Provisional application No. 60/736,797, filed on Nov. 15, 2005.
(60) Provisional application No. 60/822,440, filed on Aug. 15, 2006.
(60) Provisional application No. 60/737,287, filed on Nov. 16, 2005.

(51) Int. Cl. A61F 2/30

(52) U.S. Cl. 606/62

(57) ABSTRACT

Provided is a medical device for restoring the anatomy of bones comprising at least one inflatable device, where the inflatable device may be inflated to facilitate reconstituting the bone. Disclosed are a plurality of inflatable devices that may be used in combination in accordance with methods for reconstituting a fractured bone or other tissue.

### PRIORITY

The application claims priority from the disclosure of U.S. Provisional Patent Application Ser. No. 60/736,797, entitled “Inflatable Device For Restoring Anatomy Of Fracture Bone,” filed Nov. 15, 2005, which is herein incorporated by reference in its entirety, from the disclosure of U.S. Provisional Patent Application Ser. No. 60/822,440, entitled “Inflatable Device For Restoring Anatomy Of Fractured Bone,” filed Aug. 15, 2006, which is herein incorporated by reference in its entirety, and from the disclosure of U.S. Provisional Patent Application Ser. No. 60/737,287, entitled “Kyphoplasty Variation Bone Repair Procedure,” filed Nov. 16, 2005 which is herein incorporated by reference in its entirety.

### BACKGROUND

Versions of the present invention relate to restoring the anatomy of fractured bone and, more particularly, to restoring the anatomy of fractured bone with an inflatable device. A variety of medical devices have been created and used, but no one prior to the inventor(s) has created or used the invention described in the appended claims.

### BRIEF DESCRIPTION OF THE FIGURES

It is believed the present invention will be better understood from the following description taken in conjunction with the accompanying drawings. The drawings and detailed description that follow are intended to be merely illustrative and are not intended to limit the scope of the invention.

FIG. 1 presents a longitudinal cross-sectional view of a fractured bone shown with an inflatable device inserted therein.

FIG. 2 presents a longitudinal cross-sectional view of the fractured bone of FIG. 1 shown knitting after the insertion and inflation of the inflatable device therein.

FIG. 3a presents a longitudinal cross-sectional view of a fractured bone shown with an inflatable device having a dumbbell shape with one end of the inflatable device shown passing through one end of the bone.

FIG. 3b presents a longitudinal cross-sectional view of a fractured bone shown with one version of an inflatable device having a dumbbell shape.

FIG. 3c presents a longitudinal cross-sectional view of a fractured bone shown with one version of an inflatable device having a tapered dumbbell shape.

FIG. 3d presents a longitudinal cross-sectional view of the inflatable device of FIG. 3b shown placed within a fractured bone having a central fracture.

FIG. 3e presents a longitudinal cross-sectional view of the inflatable device of FIG. 3c shown placed within a fractured bone having a central fracture.

FIG. 4 presents a longitudinal cross-sectional view of a fractured bone shown with an inflatable device surrounding a portion of the bone.

FIG. 5 presents a longitudinal cross-sectional view of a fractured bone shown with an inflatable device having a guide or spine therein.

FIG. 6 presents a cross-sectional view taken along line 6-6 of FIG. 1 of one version of an inflatable device having multiple chambers therein.

FIG. 7 presents a side view of one version of an inflatable device having projections at both ends thereof.

FIG. 8 presents a cross-sectional view taken along line 8-8 of FIG. 7 of one version of an inflatable device having projections incorporated into the inflatable geometry of the inflatable device.

FIG. 9a presents an alternate cross-sectional view taken along line 8-8 of FIG. 7 of an inflatable device having external projections.

FIG. 9b presents an alternate cross-sectional view taken along line 8-8 of FIG. 7 of an inflatable device having internal and external projections.

FIG. 10 presents a side view of one version of an inflatable device having separately inflatable longitudinal projections extending substantially along the length of the inflatable device.

FIG. 11 presents a side view of one version of an inflatable device having longitudinal projections provided in the center of the inflatable device.

FIG. 12 presents a side view of one version of an inflatable device having projections at one end.

FIG. 13 presents a side view of one version of an inflatable device having annular rings incorporated into the surface thereof.

FIG. 14 presents a partial view of one version of an inflatable device having a plurality of inwardly projecting annular rings.

FIG. 15 presents a partial view of one version of an inflatable device having a plurality of outwardly projecting annular rings.

FIG. 16a presents a section view of one version of an inflatable device having a plurality of toothed rings angled in a first direction and a plurality of toothed rings angled in a second direction.

FIG. 16b presents a longitudinal cross-sectional view taken along line 16a-16a of the inflatable device of FIG. 16a.

FIG. 16c presents a section view of an alternate version of an inflatable device having a plurality of toothed rings angled in first direction and a plurality of toothed rings angled in a second direction.

FIG. 16d presents a longitudinal cross-sectional view taken along line 16d-16d of the inflatable device of FIG. 16c.

FIG. 17 presents a side view of one version of a threaded inflatable device.

FIG. 18 presents a side view of one version of an inflatable device having a plurality of annular rings and a plurality of longitudinal projections provided at the ends thereof.

FIG. 19 presents a side view of one version of a textured inflatable device.

FIG. 20 presents a side view of an alternate version of a textured inflatable device.

FIG. 21 presents a side view of an alternate version of a textured inflatable device.

FIG. 22 presents one version of an inflatable device configured in a dumbbell shape having texture at the ends thereof and a plurality of projections about the center, where the inflatable device is shown associated with a syringe and a delivery lumen having markings to indicate proper placement of the inflatable device in a bone cavity.

FIG. 23 presents one version of an inflatable device having randomly positioned rods configured at a plurality of angles.

FIG. 24 presents a fractured bone in need of repair having a first bone portion and a second bone portion.

FIG. 25 presents one version of a first inflatable device having a first tether inserted through the second bone portion and into the first bone portion of the fractured bone illustrated in FIG. 24.

FIG. 26 presents one version of a second inflatable device having a second tether inserted into the second portion of the fractured bone illustrated in FIG. 24.

FIG. 27 presents one version of the interaction of the first inflatable device and the second inflatable device, illustrated in FIG. 26, to provide a therapeutic effect.

FIG. 28 presents a fractured bone in need of repair having a first portion and a second portion.

FIG. 29 presents one version of an inflatable device having a first inflatable portion and a second inflatable portion, where the first inflatable portion is shown inflated in the first bone portion of the fractured bone, illustrated in FIG. 28, and the second inflatable portion is shown deflated.

FIG. 30 presents one version of the first bone portion and the second bone portion of the fractured bone, illustrated in FIG. 28, being compressed by drawing or urging the first inflatable portion of the inflatable device, illustrated in FIG. 29.

FIG. 31 presents one version of stabilizing the bone after the bone portions illustrated in FIG. 30 have been joined, where the fractured bone is shown with the second inflatable portion of the inflatable device, illustrated in FIG. 30, inflated to provide stability to the bone.

FIG. 32 presents a fractured bone having a proximal portion and a distal portion with access holes therein, where the fractured bone is shown with wedge-shaped cavities removed from each end thereof.

FIG. 33 presents one version of an inflatable device having wedge-shaped portions corresponding to the wedge-shaped cavities of FIG. 33.

FIG. 34 presents one version of the inflatable device shown in FIG. 33 being used to join fractured bone portions by applying pressure from the wedge-shaped portion of the inflatable device to the wedge-shaped cavities such that the proximal portion and the distal portion of the fractured bone are drawn together.

FIG. 35 presents a more detailed view of the relationship between the wedge-shaped portion of the inflatable device and the wedge-shaped cavity shown in FIG. 34.

FIG. 36 presents one version of an inflatable device, shown inserted into a fractured bone, having a first inflatable portion and a second inflatable portion, where the first inflatable portion is shown inflated and the second inflatable portion is shown deflated.

FIG. 37 presents one version of the inflatable device of FIG. 36 shown with both the first inflatable portion and the second inflatable portion inflated.

### DETAILED DESCRIPTION OF THE INVENTION

Versions of the present invention comprise an inflatable device for use in orthopedic procedures such as those directed towards restoring the anatomy of diseased or fractured bone. Any suitable bone, such as the humerus, may be prepped by providing a cavity therein in accordance with versions herein. Pre-existing cavities or pre-formed cavities, such as natural cavities formed within bones, may also be utilized. As will be discussed in greater detail, an inflatable device, such as a balloon, may then be inserted into the cavity. Once introduced, the inflatable device may be unfolded and/or inflated through the application of air, gas, fluid, a liquid matrix, bone paste, bone cement, bone matrix, or the like, via a lumen fluidly connected thereto. The terms “inflate” and “inflation” shall refer to distention with fluid and/or gas, an increase in volume, swelling, dilation, and/or expansion. The inflatable device may then be inflated intramedullarily with one of a plurality of lumens to apply outward pressure to the interior surface of the fractured bone. Applying pressure in the disclosed manner may provide a scaffolding upon which one or a plurality of bones may knit while the being maintained in a substantially natural shape and/or configuration.

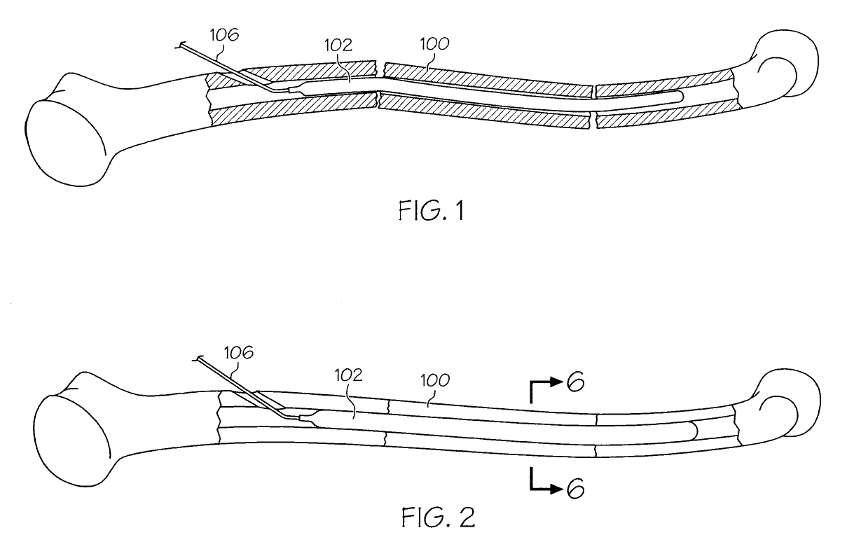

For example, referring to FIG. 1, a fractured bone 100 is shown having an inflatable device 102 inserted therein, where the inflatable device 102 is shown uninflated. FIG. 2 illustrates the fractured bone 100 of FIG. 1 after the inflatable device 102 has been inflated, where inflation of the inflatable device 102 has substantially restored the geometry of the fractured bone 100. The inflatable device 102 may be a single layer inflatable device, such as a balloon of predetermined size and/or shape, configured to conform to the internal cavity created in the bone 100 and to restore original anatomical shape and position of the bone 100. For example, collapsed fractures of cortical bone may be treated by restoring the bone from an intramedullary approach. Inflation of the balloon with a fluid, gas, materials disclosed in greater detail herein, or the like, may take place via a delivery lumen 106. Internal cavities in bone may be created using any suitable cutting technique or tool.

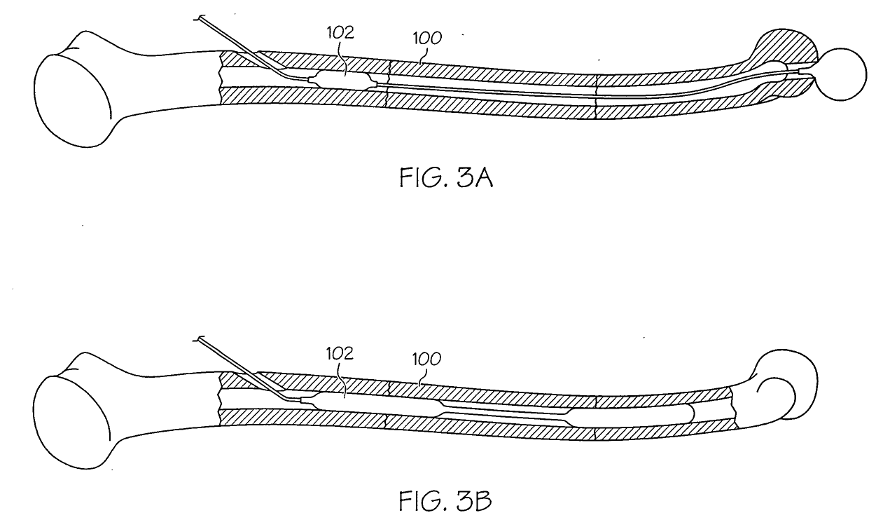

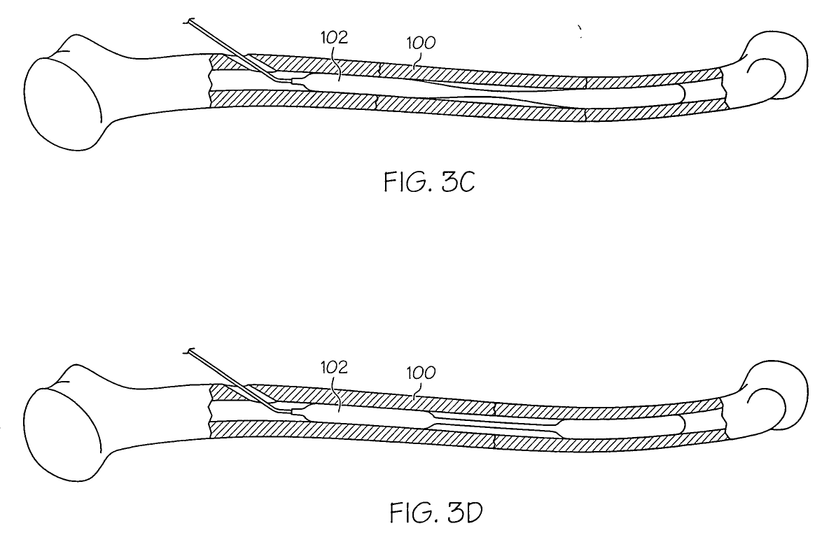

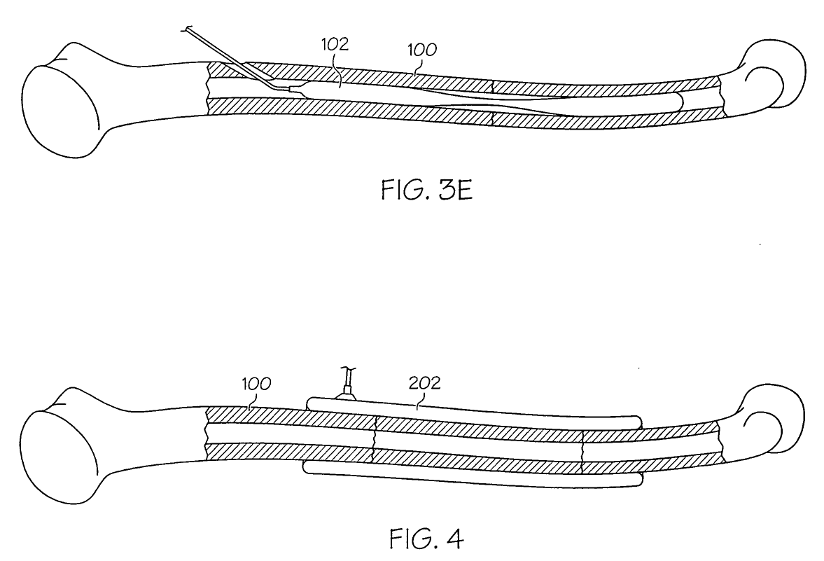

In one version, the inflatable device 102 may be any suitable device having elastomeric qualities, or partially elastomeric qualities, such as, for example, a balloon. The inflatable device may be provided associated with the fractured bone in any suitable mode or manner such that a therapeutic effect is achieved. For example referring to FIG. 3a, a through all hole in a bone 100 may be provided through which an inflatable device is inserted such that the distal end of the inflatable device passes through the bone and is positioned and inflated outside the distal end of the bone. The distal end of the inflatable device may be configured to inflate to a diameter greater than the through all hole where, after inflation, proximal tension may be applied to the inflatable device to tension the bone and provide therapeutic compression. It is further contemplated that an expandable portion of the inflatable device may be inflated within the bone to provide an outward pressure to stabilize the bone and to prevent fractured bone portions from collapsing upon each other.

As illustrated, for example, in FIGS. 3b-3e, the inflatable device 102 may be provided with multiple or one of a plurality of geometries such as, for example, a longitudinal shape, a dumbbell shape, a tapered dumbbell shape, a necklace shape having a plurality of in-line spheres, a circular shape, a collar shape, an annular shape, any other suitable shape or configuration, or combinations thereof. Additionally, the inflatable device 102, or portions thereof, may be constructed from, or contain, a fibrous material configured to retain a desired geometry where, for example, increasing pressure within the inflatable device increases the rigidity of the device without substantially altering the shape of the inflatable device 102. The inflatable device 102, or portions thereof, may be made of a composite material having the flexibility and elastic characteristics of an elastomeric material, yet exhibit the growth limits of inelastic materials. Additionally, the inflatable device 102 may be placed in any suitable tissue such as a long bone having a single fracture, a long bone having a compound fracture, vertebrae, or any other suitable bone.

Referring to FIG. 4, the balloon or inflatable device 202 may be applied to the outer surface of the bone 100 to assist in healing a fracture, or the like. One or a plurality of inflatable devices 202 may be wrapped around, or otherwise associated with, the outer surface of the bone to target, for example, particular fractures in a bone. The inflatable device 202 may have a geometry tailored to mend the bone and to match the desired bone shape. The inflatable device 202 may be pressurized such that pressure is applied inward against the outer surface of the bone to mend fractures or the like. Providing an outer collar, for example, may provide a minimally invasive or non-invasive mode of healing damaged bones. In one version, a balloon applied to the outer surface of a bone may be used in combination with an inflatable device, such as the inflatable device 102 illustrated in FIGS. 1-3e, positioned within the bone, where the internal balloon may be inflated to exert outward pressure and the external balloon may be inflated to exert pressure inward to provide a therapeutic effect.

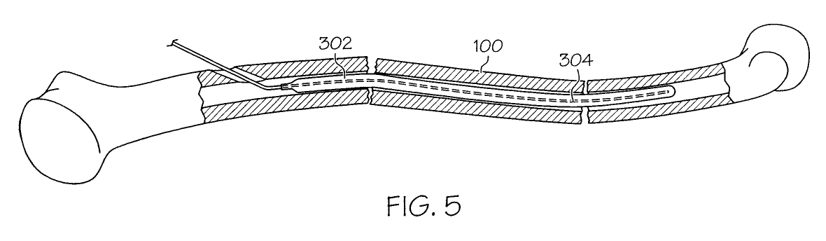

Referring to FIG. 5, in one version, the inflatable device 302 may be integrated with a rod or other spine member 304 to add support and/or guidance. The spine member 304 may be affixed to the inflatable device 302, integral with the inflatable device 302, flush with the inflatable device 302, projecting from the inflatable device 302, and/or otherwise suitable configured. The spine member 304 may be a central spine operably configured increase rigidity and to support compressive loads, tensile loads, bending loads, shearing loads, torsional loads, and rotational loads. Alternate versions of projections and rods are disclosed in more detail herein. It is contemplated that any suitable shape or configuration of inflatable device may be provided in accordance with versions herein. It is further contemplated that the inflatable device may have projections therefrom, such as inflatable or solid protrusions, which may be used to grasp, hold, and/or secure portions of the bone.

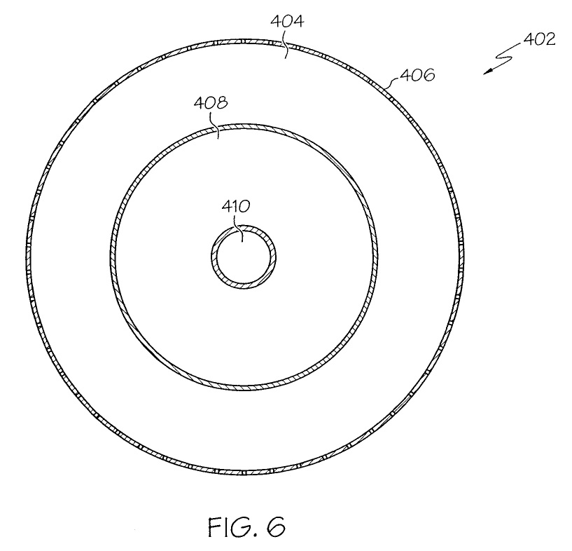

Referring to FIG. 6, a cross-sectional view of an inflatable device 402 is shown taken along line 6-6 of FIG. 2. The balloon or inflatable device 402 may have multiple layers, lumens, tubes, cavities, or the like to provide desired surface characteristics, resistance to puncture and tearing, or other beneficial properties. Additionally, the inflatable device may be provided with multiple discrete chambers, cavities, lumens, tubes, or the like adapted to perform various functions. For example, as illustrated, the inflatable device 402 includes an outer chamber 404 having a porous outer surface 406 that is connected to a delivery lumen (not shown), where an adhesive or the like may be administered through the delivery lumen into the outer chamber 404. The addition of an adhesive, bone matrix, bone paste, bone cement, synthetic paste, therapeutic agent, healing agent, structural agent, or other suitable material, may assist or speed the healing process, assist in fitting the balloon properly, provide a dye or visual marker or the like to visually identify the position of the balloon in a bone through scans or x-ray, provide structural support, or serve any other suitable purpose. It will be appreciated that the outer chamber 404 is disclosed by way of example only, where any suitable number of chambers for any suitable purpose are contemplated.

For example, in an alternate version, the outer chamber may include one or a plurality of rods, bands, support members, stabilizers, projections, or the like that may be inflated via an independent delivery lumen. Inflating the projections, or the like, of an inflatable device may allow a user to tailor the amount of rigidity, stability, texture, or the like, of the inflatable device desirable for a given procedure. The pressure of the projections or texture may, for example, be controlled independently from the inflation of the inflatable device.

A second chamber 408, connected to a delivery lumen (not shown), may be provided and configured to pressurize the bone in an intramedullary fashion. A third chamber 410, connected to a delivery lumen (not shown), may be provided and configured to receive a liquid capable of solidifying within the bone to restore the original anatomical shape or size thereof. The first chamber 404, the second chamber 408, and the third chamber 410 may be used in combination to effectively treat and repair fractured bones. It will be appreciated that the illustrated chambers 404, 408, 410 are disclosed by way of example only and may be tubes, lumens, cavities, portions, inflatable elements, or the like and that any suitable number of configurations of chambers may be provided. For example, the inflatable device may have two chambers, three chambers, four chambers, five chambers, six chambers, or any other suitable number where, for example, each chamber may have a designated function.

Once the fractured bone has been sufficiently restored, the one or a plurality of balloons, inflatable devices, lumens, or the like may be deflated and/or removed, may be left inside the bone to degenerate, may be left in the bone as part of the new bone growth, or may otherwise be managed to provide a therapeutic effect. The inflatable device may be provided with luminous sections and/or regions of porosity for the delivery of therapeutic agents, bonding agents, or the like, and may be treated to exhibit regions of distinct compliance. In one version, a multi-luminal tube is operably configured to facilitate the insertion of therapeutic or bonding agents at, for example, the same rate that the balloon is being deflated such that the anatomical position and shape of the treated bone are substantially maintained where, for example, the third chamber 410 is filled with a liquid cement as the second chamber 408 is deflated.

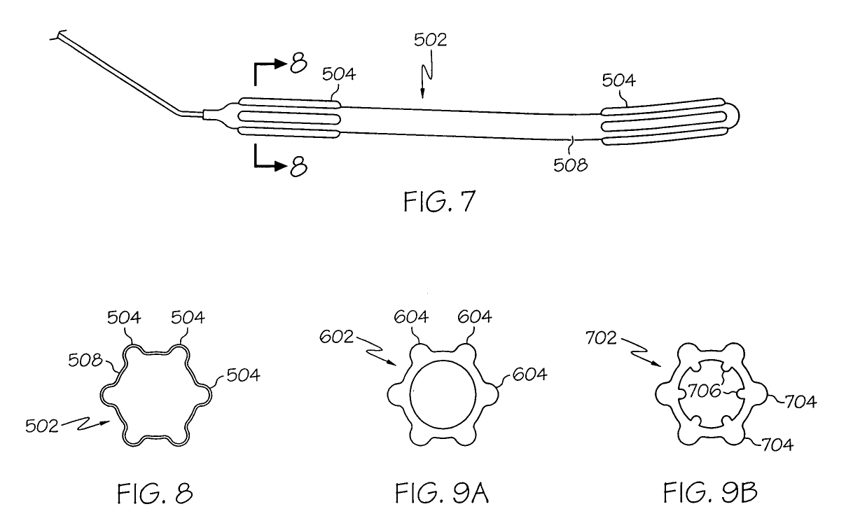

Referring to FIGS. 7-23, versions of the inflatable device may have projections or the like extending therefrom to reduce the torsional and/or axial movement of the inflatable device during placement and/or to prevent slippage of the balloon or inflatable device. Referring to FIG. 7, an inflatable device 502 may include longitudinal ridges or projections 504 projecting laterally therefrom to provide torsional and/or longitudinal rigidity to the balloon, torsional and longitudinal stability to the bone, and/or to increase the friction coefficient of the balloon when it engages the inner surface of a bone. As illustrated, the projections 504 may be placed at each end of the balloon, or at any other suitable location.

Referring to FIG. 8, shown is a cross-sectional view of the inflatable device 502 of FIG. 7. As illustrated, the projections 504 may be made from the geometry of the balloon surface when inflated. The outer surface 508 of the inflatable device 502 may take on a textured shape or the shape of a support member upon inflation, where portions of the balloon may function as longitudinal support members, annular support members, woven support members, or any other configuration of support member or texture inflated in association with the inflation of the inflatable device.

Referring to FIG. 9a, a cross-sectional view of an alternate version of an inflatable device 602 is shown having projections 604 that may be configured from hardened or shape retention material and may function as longitudinal support members, annular support members, woven support members, or any other configuration of support member or texture. The projections 604, ridges, extensions, protuberances, surface changes, material changes, and/or texture may be configured from material that maintains the general shape of the projections or the like in the absence of inflation. The projections 604, or the like, may be formed from hardened materials, semi-rigid materials, rigid material, memory retention material, or any other suitable material.

Referring to FIG. 9b, one version of an inflatable device is depicted having both internal projections 706 and external projections 704. Providing both internal projections 706 and external projections 704 may further increase the axial and torsional rigidity of the inflatable device 702. It will be appreciated that the projections, support members, or the like, may be integrated into the surface of the inflatable device, may be lateral outward projections, may be inward projections, or may have any other suitable configuration to improve the torsional rigidity and axial or longitudinal rigidity of the balloon, the longitudinal and torsional stability of the bone, and/or to diminish the slippage of the inflatable device.

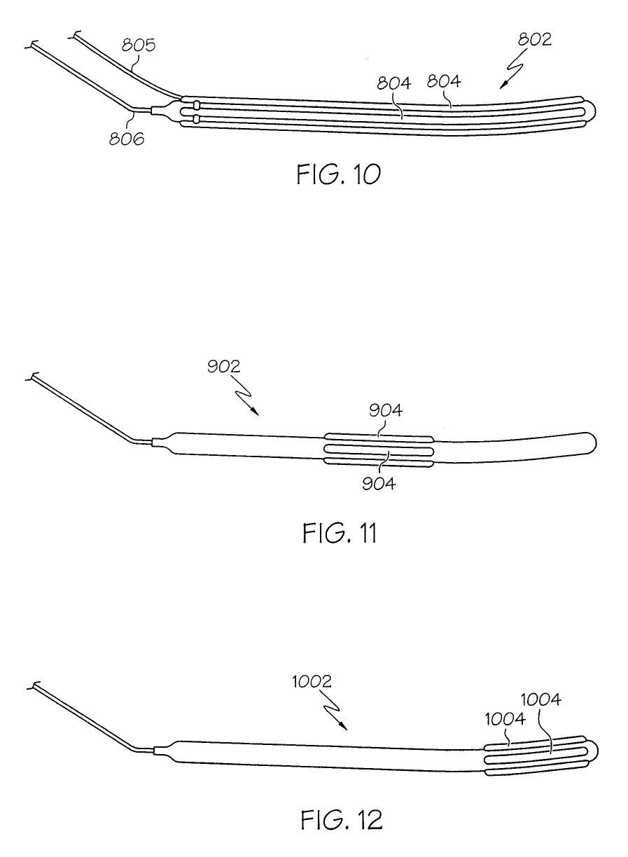

Referring to FIGS. 10-12 the torsional rigidity and the longitudinal or axial rigidity of the inflatable device may be improved by incorporating rods or tubes, such as semi-rigid longitudinal rods, into all or a portion of the inflatable device. The rods or tubes may extend longitudinally along the length or along a portion of the inflatable device and may be placed at more specific areas where longitudinal motion, torsion, and/or torsional slipping are more likely to occur. The projections or rods may be solid, may be inflatable, may be independently inflatable from the inflatable device, or have any other suitable configuration.

Referring to FIG. 10, an inflatable device 802 is shown having a delivery lumen 806 associated therewith and a plurality of projections 804, where the projections 804 are independently inflatable via a delivery lumen 805. With reference to all versions herein, the rods or tubes may be made of any suitable material such as balloon material, semi-rigid material, short segments of rigid material, tacky material, memory retention material, adhesive material, rigid material, elastomeric material, and/or any other suitable material. In one version, the rods, tubes, projections, texture or the like are inflatable via a lumen separate from that of the inflatable device. The tubes, texture, rods, or the like may then be pressurized or sized via the associated lumen to a desirable pressure, size, configuration, shape, or the like. Referring to FIG. 11, an inflatable device 902 is shown having projections 904 positioned at about the center thereof. Referring to FIG. 12, an inflatable device 1002 is shown having projections 1004 at about the distal end thereof.

The rods or projections, which include tubes, bars, spines, protuberances, extensions, support members, combinations thereof, or the like, may be inserted into, attached to, affixed to, coupled with, or formed integrally with the inflatable device in a linear configuration, in a non-linear configuration, in an annular configuration, in a lateral configuration, in a longitudinal configuration, in a wave-shaped configuration, in a random configuration, in a non-linear configuration, in a threaded configuration, and/or in any other suitable configuration. The inflation of the rods and other projections may be independent of the inflatable device or integral with the inflatable device. The projections or the like may project in any suitable direction or manner, such as outwardly from the inflatable device, as shown in FIG. 7, or inwardly towards the centroid of the balloon, as shown in FIG. 9b.

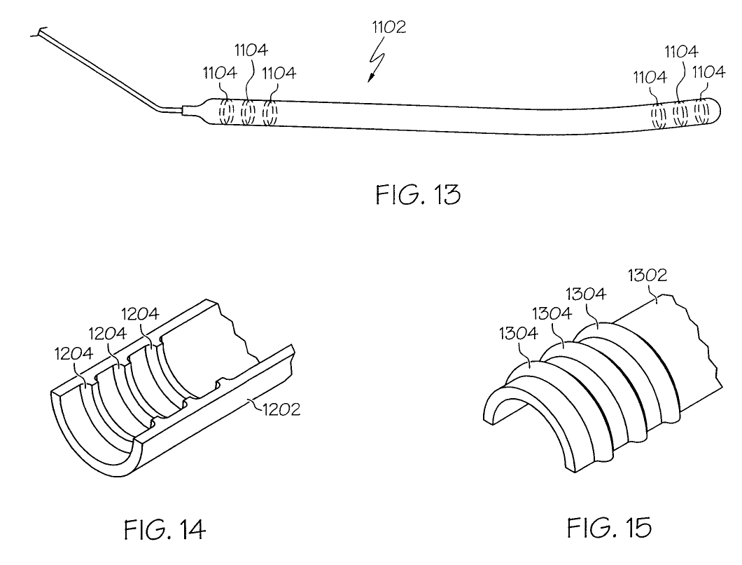

Referring to FIG. 13, an alternate version of an inflatable device 1102 is shown where the torsional rigidity and longitudinal rigidity of the balloon may be increased by altering the material characteristics of portions of the balloon. Portions of the inflatable device 1102 may be provided with hardened annular bands 1104 flush with the inflatable device 1102 to provide torsional and/or longitudinal rigidity and/or stability. The annular bands 1104 may be rigid, semi-rigid, deformable with memory retention capabilities, or have any other suitable configuration. It will be appreciated that any suitable portions, such as longitudinal strips or bands, of an inflatable device may be hardened or otherwise providing with modified material characteristics. The annular bands may be contiguous, intermittent, or have any other suitable configuration.

Referring to FIG. 14, adding annular band projections 1204 to an inflatable device 1202 that project inwardly may improve the torsional and longitudinal rigidity of the inflatable device and the torsional and longitudinal stability of the bone. The annular band projections 1204 may be configured from materials and in a manner similar to the longitudinal support members described herein. The annular band projections 1204 may be a separate inflatable device associated with a balloon, where the annular band projections may be inflated with a dedicated delivery lumen (not shown). The annular band projections 1204 may be tapered, beveled, rounded, or provided with any other suitable configuration.

Referring to FIG. 15, adding annular band projections 1304 to an inflatable device 1302 that project outwardly may improve the torsional and longitudinal rigidity of the inflatable device and the torsional and longitudinal stability of the bone. The annular band projections 1304 may be configured from materials and in a fashion similar to the longitudinal support members described herein. The annular band projections 1304 may be a separate inflatable device associated with a balloon, where the annular band projections may be inflated with a dedicated delivery lumen (not shown). It will be appreciated that an inflatable device may be provided with both internally and externally projecting annular band projections. It will be further appreciated that each of the annular bands may be a contiguous annulus or band, an intermittent band, or any other suitable configuration.

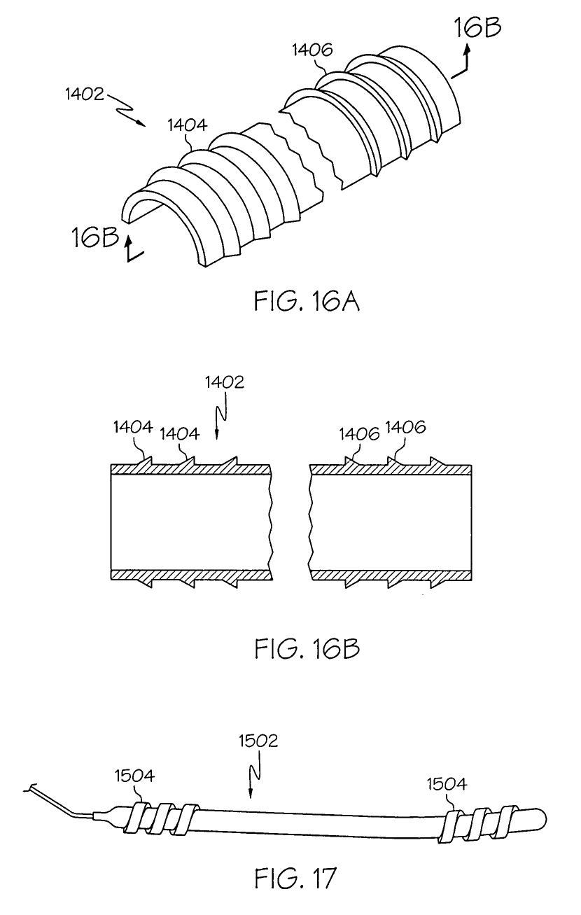

Referring to FIGS. 16a-16d, an inflatable device 1402 may be provided with ridges, teeth, or the like. As illustrated, the ridges may be angled teeth having a ratchet-like function such that upon insertion the inflatable device is resistant to movement or removal. The ridges may point in the same direction, in opposite directions at opposite ends, as illustrated by ridges 1404 and ridges 1406, in a random configuration, in a threaded configuration, in an annular configuration, in a longitudinal configuration, in a contiguous configuration, in an intermittent configuration, and/or in any other suitable configuration. The ridges 1404 and ridges 1406 may maintain fractured bone portions in proximity to one another, yet also may provide resistance against the bone portions collapsing inward upon each other.

Referring to FIG. 17, an inflatable device 1502 may be provided with one or a plurality of threaded projections 1504. The threaded projections 1504 may extend internally and/or externally and may increase the torsional and/or axial rigidity of the inflatable device. In one version, the inflatable device may be provided with a rod, tube, material property, or the like, flush with the surface thereof to increase the torsional or axial rigidity.

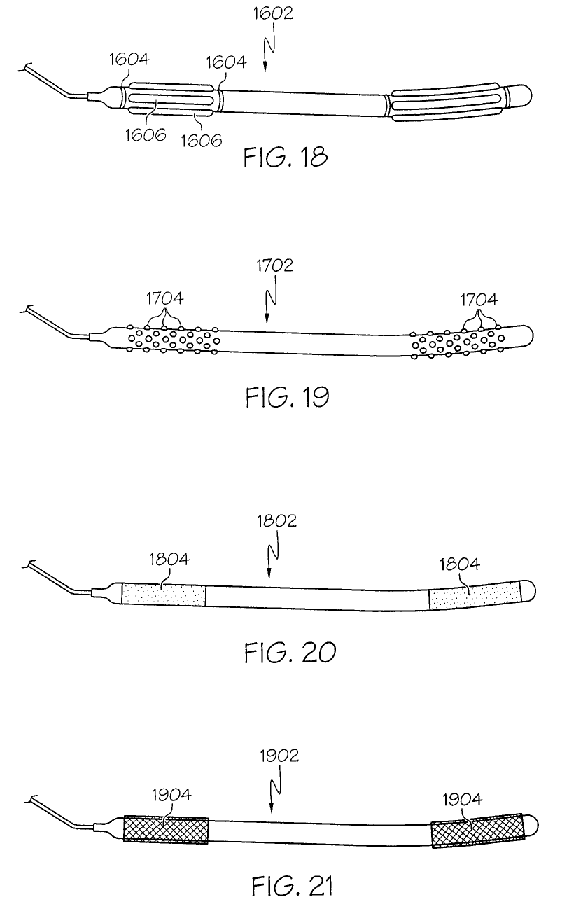

Referring to FIG. 18, in one version of an inflatable device 1602 a plurality of annular bands 1604 may be combined with a plurality of rods 1606 to improve the torsional and longitudinal rigidity of the inflatable device and/or to reduce slippage on the bone. It will be appreciated that any combination of texture, projections, support, configuration, shape, size, or the like of embodiments disclosed herein is contemplated.

Referring to FIGS. 19-23, the surface of a balloon or inflatable device may be textured or otherwise modified or altered to decrease slippage and to prevent torsion or axial movement of at least a portion of the balloon. Texture or surface effect may also increase the rigidity of the balloon. The surface texturing may be combined with or may include rods, support members, annular bands, tubes, projections, or the like, to increase the rigidity and reduce the slippage of the inflatable device.

Surface texture or a surface effect may be accomplished with attachments, projections, additives, protuberances, extensions, or the like from the balloon surface. The projections may be, for example, affixed pieces of plastic, injection molded projections, particles sprayed on with an adhesive, an abraded surface, or the like, where the texture or surface effect may otherwise be affixed to, inserted into, created on, or constructed integrally with the balloon.

The projections, for example, may be circular in shape, rod-like in shape, amorphous in shape, symmetrical in shape, asymmetrical in shape, of varying size, of varying shape, of uniform size, of uniform shape, of varying height, of uniform height, or combinations thereof. Referring to FIG. 19, surface texture 1704 may be provided to an inflatable device 1702 at any suitable location, including those areas where slippage is likely to occur, where bending strength is most needed, and/or where torsional and axial rigidity is most desirable, such as at about the ends of the inflatable device 1702.

Referring to FIG. 20, the surface effect or texture 1804 of a portion or all of the inflatable device 1802 may be particulate or granular to prevent or reduce slippage of the inflatable device 1802. For example, sand, granular, or particulate matter may be incorporated into the surface of the balloon at both ends thereof. The particulate may be manufactured integrally with the inflatable device, may be affixed by spraying on the particulate with an adhesive, or may otherwise be suitably attached. It will be appreciated that versions illustrated herein may be combined to provide a desirable combination of torsional rigidity, longitudinal rigidity, and/or reduced slippage.

Referring to FIG. 21, the texture or friction coefficient of the balloon may be increased with a woven portion, tread portion, raised portion, and/or patterned portion 1904 present on at least a portion of the surface of the inflatable device 1902. The raised or patterned portion 1904 of the balloon may be configured from the same material as the inflatable device 1904, a semi-rigid material, a thread-like material, a fibrous material, a memory retention material, a rigid material, or any other suitable material to provide the inflatable device 1902 with a woven or patterned tread. The patterned portion 1904 of the inflatable device 1902 may be created integrally with the balloon upon construction, may be added to the balloon at a second time, may be woven around the inflatable device, may be affixed with an adhesive, and/or may otherwise be suitably attached. It will be further appreciated that various materials having different friction coefficients may be combined to reduce slippage of the inflatable device 1902 at, for example, different locations on the inflatable device 1902. For example, particulates of different grades may be utilized at various locations, different patterns may be utilized, different combinations of projections and particulates may be utilized, or any other suitable combination.

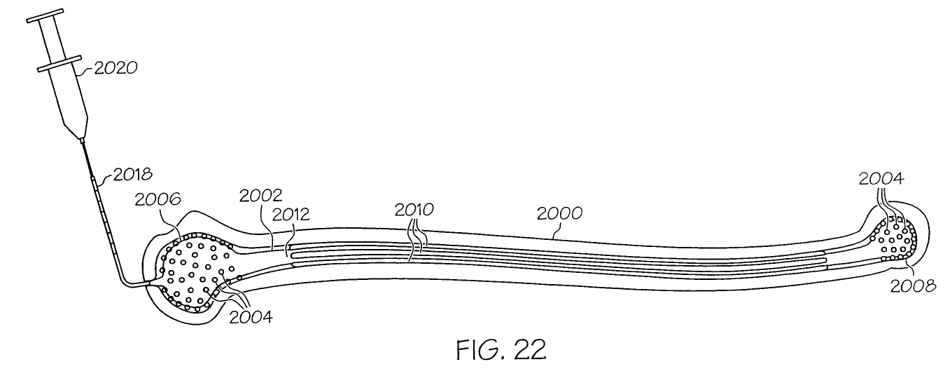

Referring to FIG. 22, the inflatable device 2002 or balloon may be any suitable shape including, as illustrated, a dumbbell-shaped balloon. It is further contemplated that the inflatable device or balloon shapes may be linear, amorphous, symmetrical, tapered, tapered dumbbell, pearl necklace-shaped, shaped to correspond to a particular anatomy, curved, round, spherical, or any other suitable shape. The inflatable device 2002 may include, for example, texture, projections, or protuberances 2004 on a first dumbbell portion 2006 and a second dumbbell portion 2008 of the inflatable device 2002 to prevent slippage of the inflatable device 2002. In addition to the texture, projections, or protuberances 2004, the inflatable device 2002 may include rods or support members 2010, such as along the central portion 2012 of the inflatable device 2002, to increase the torsional rigidity, axial rigidity, and bending strength of the inflatable device 2002.

Still referring to FIG. 22, the inflatable device 2002 may be inflated with any suitable inflation device 2020, such as a syringe, via a delivery lumen 2018, where fluid, air, gas, gel, hardening agents, scaffolding agents, adhesive agents, therapeutic agents, dye agents, and/or any other suitable substance may be injected or otherwise inserted into or around the inflatable device 2002. A tube or delivery lumen 2018 may connect the syringe or inflation device 2020 to the inflatable device 2002, where the delivery lumen 2018 may be provided with incremental markings to indicate how far the inflatable device 2002 has been inserted into a bone 2000 in order to determine proper placement. For example, a series of lines may be provided along a tube or lumen to indicate when the distal end of the balloon has been inserted into the distal end of the bone cavity.

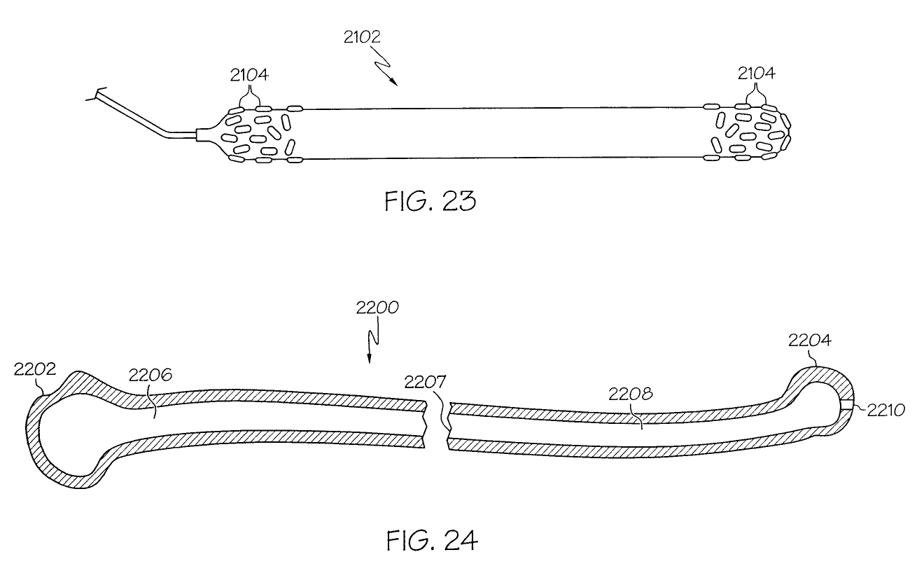

Referring to FIG. 23, disclosed is one version of a longitudinal inflatable device 2102 having a plurality of projections 2104 positioned thereon. The projections 2104 may be rods of substantially identical or similar shape positioned at random about portions of the inflatable device 2102, such as at the ends thereof, to increase torsional rigidity, longitudinal rigidity, and/or to prevent slippage. In one version, such projections 2104 may be placed in a pre-selected pattern, where the location of the projections 2104 on the inflatable device 2102 is uniform, but the rotational orientation of each of the projections 2104 is selected randomly such that the projections 2104 generally point in a variety of directions. Alternate versions include providing any suitable shape such as star shapes, cross shapes, or spirals.

Referring to FIGS. 24-27, one version of a system and method for healing a fractured bone 2200 is disclosed. FIG. 24 illustrates a fractured bone 2200 split into a first portion 2202 and a second portion 2204. The illustrated bone 2000 depicts a first cavity 2206 and a second cavity 2208 that may be created through an access point 2210 at one end of the bone, via access points at both ends of the bone, or by any other suitable device, method, mechanism, or mode at any suitable location. One or a plurality of cavities having any suitable shape or configuration may be created in the fractured bone prior to insertion of one or a plurality of inflatable devices. The cavities may include one or a plurality of central bores through, for example, long bones.

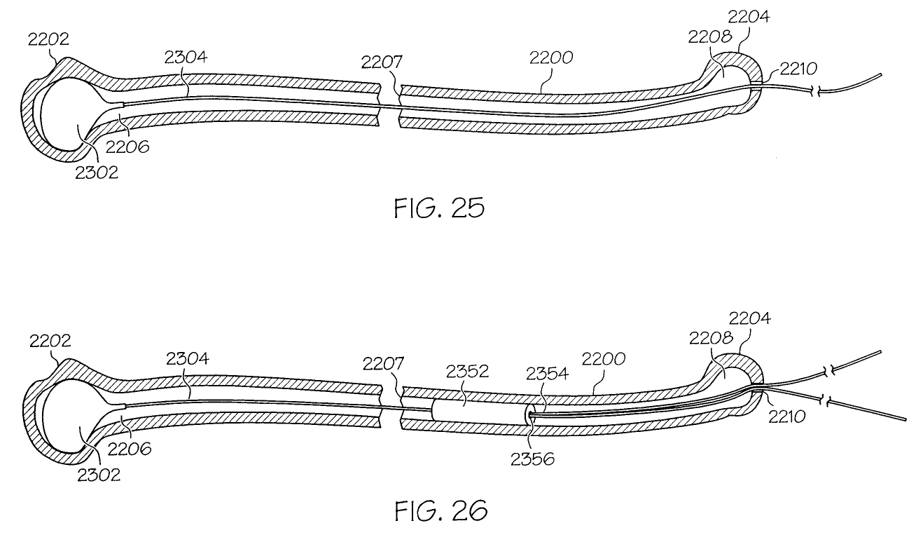

FIG. 25 illustrates one version of a first inflatable device 2302 that is inserted through an access point 2210 in the second portion 2204 of the fractured bone 2000 into a cavity 2206 of the first portion 2202 of the fractured bone 2200. The first inflatable device 2302 may include a first tether 2304 extending proximally therefrom that extends back through the access hole 2210 and out of the body for manipulation by a clinician. The first tether 2304 may be a tube, a cannulated member, a flexible cord, a rigid rod, a semi-rigid rod, a guide, or any other suitable connection. The first tether 2304 may be a delivery lumen, a support rod, an actuator, an indicator of position or placement, and/or any other suitable device.

The first inflatable device 2302 may be inserted into the first portion 2202 of the fractured bone 2000 in a deflated state where, upon placement and positioning, the first inflatable device 2302 is inflated to a size greater than the access point 2210 in the second portion 2204 of the bone or to a size sufficiently large to substantially secure the first inflatable device 2302 within the bone. The inflation of the first inflatable device 2302 may secure the first inflatable device 2302 within the first bone portion 2202 by pressing against the inside surface of the bone.

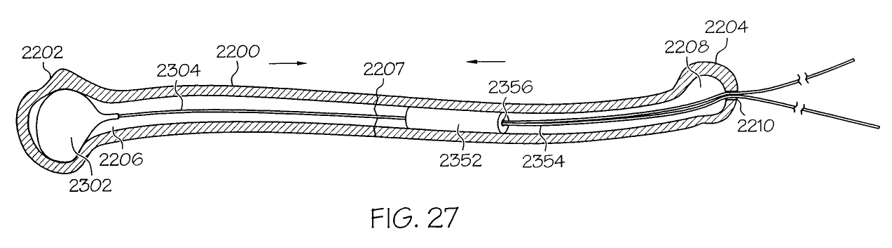

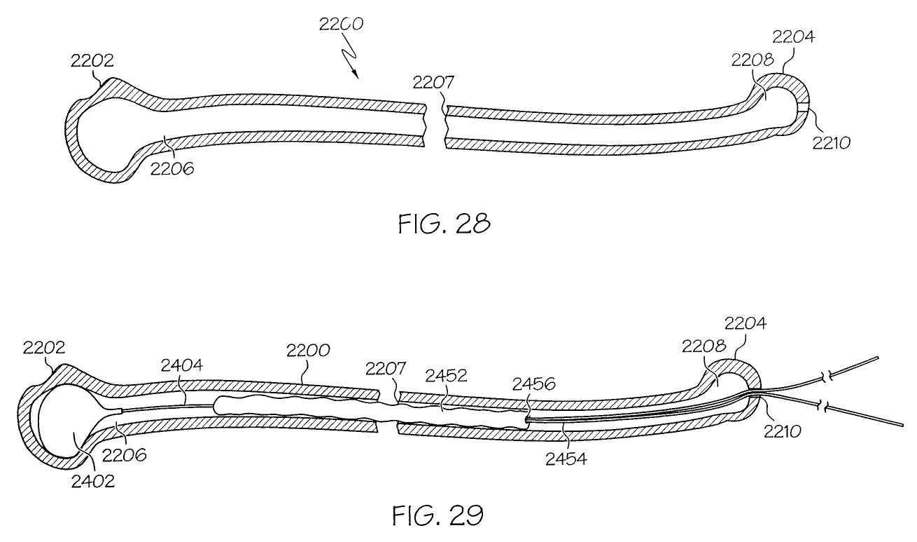

Referring to FIG. 26, after placement of the first inflatable device 2302, a second inflatable device 2352 may be inserted into the cavity 2208 of the second bone portion 2204 through the access point 2210. The second inflatable device 2352 may have a second tether 2354 associated therewith that passes proximally through the access point 2210 and out of the body for manipulation by a clinician. The second tether 2354 may be a tube, a cannulated member, a flexible cord, a rigid rod, a semi-rigid rod, a guide, or any suitable connection and may include a delivery lumen for inflation. The second inflatable device 2354 may include a passageway 2356, such as a central bore, through which the first tether 2304 of the first inflatable device 2302 may pass. The first tether 2303 of the first inflatable device 2303 may act as a guideline along which the second inflatable device 2352 may be inserted into the fractured bone 2000.

The second inflatable device 2352 may be placed at any location along the fractured bone 2200 such as, for example, forward of the fracture 2207, at about the fracture 2207, or rearward of the fracture 2207. Upon insertion and placement, such as in the second bone portion 2204, the second inflatable device 2352 may be inflated such that it is retained within the cavity 2208. The inflation of the second inflatable device 2352 may secure the second inflatable device 2352 within the second bone portion 2204 by pressing against the inside surface of the bone and/or by inflating to a size greater than a reduced diameter portion of the bone. It will be appreciated that any of the disclosed steps may be performed in any suitable order where the second inflatable device, for example, may be placed prior to the placement of the first inflatable device.

Referring to FIG. 27, one method of rejoining the first and second bone portions 2202, 2204 of the fractured bone 2200 is disclosed. After inflating both the first inflatable device 2302 and the second inflatable device 2352, the first inflatable device 2302 may be drawn proximally, in the direction of the access point 2210, with the first tether 2304 towards the second bone portion 2204. The second bone portion 2204 may be held static or pushed proximally with the second tether 2354 to facilitate reconnection. By manipulating the bone portions 2202, 2204 with the first and second tethers 2304, 2354, respectively, the clinician may rejoin the bone portions to encourage knitting. It will be appreciated that the tethers 2304, 2354 may be tensioned or otherwise configured or manipulated to restrict the bone portions from collapsing upon one another or otherwise improperly healing.

To join the fractured bone portions 2202, 2204, it will be appreciated that the first bone portion 2202 may be held substantially still while the second bone portion 2204 is urged distally, the first bone portion 2202 may be pulled proximally while the second bone portion 2204 is pushed distally, or the first bone portion 2202 may be pulled proximally in the direction of the access point 2210 while the second bone portion 2204 is held substantially still. The tethers 2304, 2354 may be adjusted or manipulated by the clinician such that the desired fit and/or compressive force between the bone portions 2202, 2204 is achieved, where the force may be sufficient to reconnect the bone portions 2202, 2204 but not over compressed such that the bone portions 2202, 2204 collapse upon each other. It will be appreciated that the illustrated example discloses two portions of a fractured bone by way of example only, where a plurality of inflatable devices may be used to rejoin and/or compress a plurality of bone portions in a compound or multi-piece bone fracture. It will be appreciated that methods of reconnection disclosed herein may include the incorporation of texture, projections, or the like into the inflatable devices.

Referring to FIGS. 28-31, an alternate version of a system and method for healing a fractured bone 2200 is disclosed. FIG. 28 illustrates a fractured bone 2200 split into a first bone portion 2202 having a first cavity 2206 and a second bone portion 2204 having a second cavity 2208 provided therein. The cavities 2206, 2208 may be provided by any suitable mode, method, or device. It will be appreciated that pre-cut cavities may not be provided where, for example, the device may be directly inserted into a portion of a bone or existing bone cavities may be used.

FIG. 29 illustrates one version of a first inflatable device 2402 that is inserted through an access point 2210 in the fractured bone 2200 into a cavity 2206 of the fractured bone 2200. The first inflatable device 2402 may include a first tether 2404 extending proximally therefrom that extends back through the access hole 2210 and out of the body for manipulation by a clinician. The first tether 2404 may be a tube, a cannulated member, a flexible cord, a rigid rod, a semi-rigid rod, a guide, and/or any other suitable connection. The first inflatable device 2402 may be inserted into the first bone portion 2202 of the fractured bone 2200 in a deflated state where, upon placement and positioning, the first inflatable device 2402 is inflated to a size greater than a region of the bone having a lesser diameter. The inflation of the first inflatable device 2402 may secure the first inflatable device 2402 within the first bone portion 2202 by pressing against the inside surface of the bone and/or by inflating to a size greater than the access point 2210 to the first bone portion 2202. Surface effects, configuration of the inflatable device, and/or the material provided may facilitate securing the inflatable devices within the bone.

In an alternate version, the first inflatable device may be configured for placement within the shaft of a bone, such as where the first inflatable device has a cylindrical configuration (not shown), where inflation of the cylinder may exert pressure on the interior surface of the bone for stability or movement. It will be appreciated that any inflatable devices or balloons disclosed and described in accordance with methods herein is by way of example only where, for example, a balloon shown inflated within a cavity may be replaced by a balloon inflated along a bone shaft, or the like, and vice versa.

Still referring to FIG. 29, a second inflatable device 2452 is provided that may be used in cooperation with the first inflatable device 2402. The second inflatable device 2452 may be placed in proximity to, coupled with, or affixed to the first inflatable device 2402. The second inflatable device 2452 may include a second tether 2454 for manipulation and/or inflation and may include a passageway 2456, such as a central bore, therethrough to accommodate the first tether 2404. The second inflatable device 2452 may be an elongated balloon positioned proximal to the first inflatable device 2402 in the direction of the access point 2210. The second inflatable device 2452 may be positioned forward of the bone fracture 2207, at about the fracture 2207, or rearward of the fracture 2207.

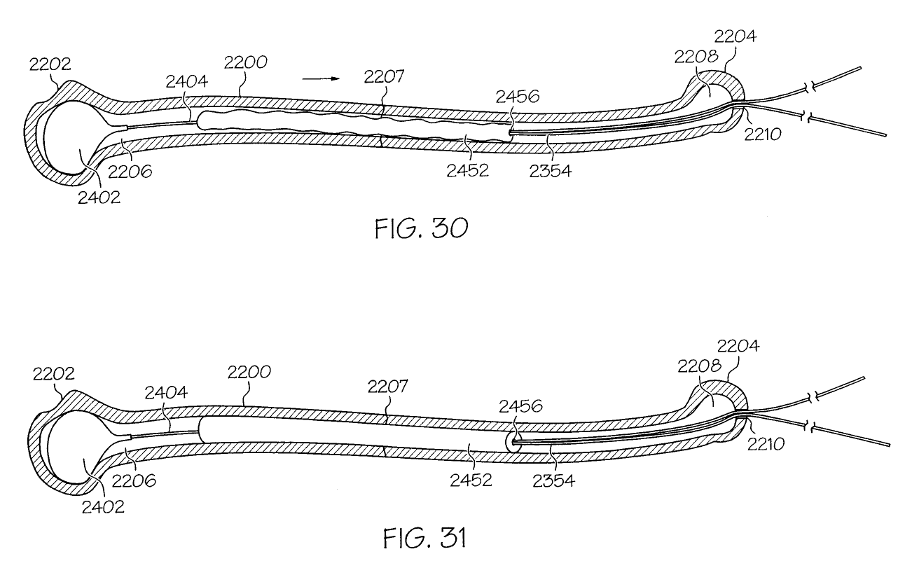

Referring to FIG. 30, the first bone portion 2202 of the fractured bone 2200 may be drawn by the first tether 2404 towards the second bone portion 2204. Once rejoined, the first tether 2404 may be used to provide compressive force between the bone portions 2202, 2204. As with all compressive loading versions herein, the tether may be secured such that compressive loading of the bone portions is maintained to sustain a therapeutic level for knitting without overloading or over compressing the bone coupling such that the portions collapse upon each other.

Referring to FIG. 31, the fractured bone 2200 may be further encouraged to heal by inflating the second inflatable device 2452 after the fractured bone portions 2202, 2204 have been joined. The second inflatable device 2452 may be inflated via the second tether 2454 associated therewith such that pressure is applied outwardly against the inner surface of the bone. The longitudinal compressive load provided by the first inflatable device 2402 and first tether 2404, in combination with the stabilizing force provided by the second inflatable device 2454, may serve to speed the healing process of the fractured bone 2200 and may encourage the bone to mend in a manner substantially consistent with the original anatomy of the bone.

Upon healing or joining of the fractured bone 2200, the first and second inflatable devices 2402, 2452 may be deflated, the inflatable devices 2402, 2452 may be removed from the bone, the inflatable devices 2402, 2452 may be left as they were during the healing process, and/or the inflatable devices 2402, 2452 or cavities 2206, 2208 may be filled. The inflatable devices 2402, 2452 or cavities 2206, 2208 may be filled with cement, bone matrix, bone paste, synthetic paste, hydroxy appetite, PMMA, a balloon inflated with fluid or air, materials disclosed herein, and/or any other suitable material. In the alternative, the inflatable devices 2402, 2452 or cavities 2206, 2208 may be left unfilled. It will be appreciated that any suitable number of inflatable devices configured to provide a compressive force and inflatable device configured to provide stabilization may be combined depending on the anatomy of a fracture or the needs of a procedure. It will be further appreciated that the method and system disclosed in FIGS. 24-27 may be used in combination with the method and system disclosed in FIGS. 28-31 to effectively mend fractured bones.

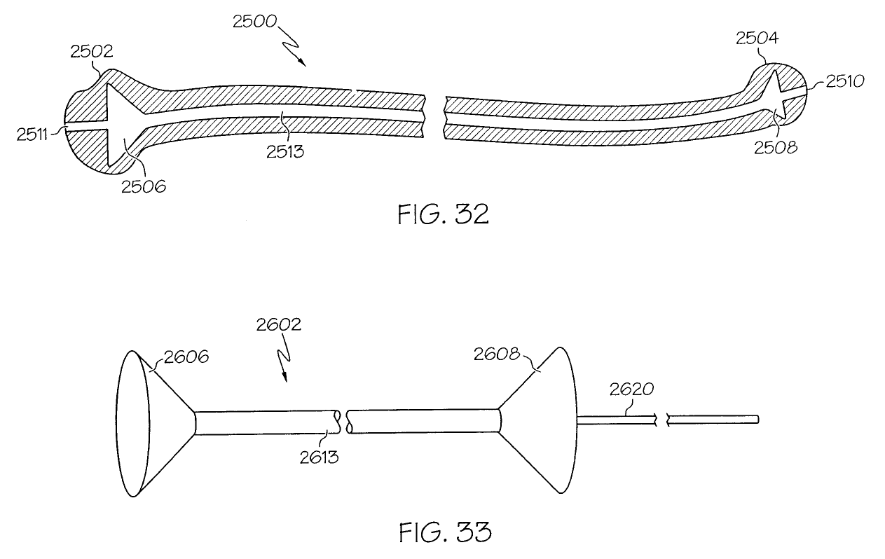

Referring to FIG. 32-34, one version of a system and method for healing a fractured bone 2500 is disclosed. FIG. 32 illustrates a fractured bone 2500 split into a first bone portion 2502 having a first wedge shaped cavity 2506 and a second bone portion 2504 having a second wedge shaped cavity 2508 formed therein. In the illustrated version, the first and second bone portions 2502, 2504 of the fractured bone 2500 have been provided with wedge shaped cavities 2506, 2508 via access points 2510, 2511, respectively, where the wedge-shaped cavities 2506, 2508 may be provided by any suitable cutting instrument and may be joined by a hollow bore 2513. It will be appreciated that the cavities may be formed via one or a plurality of access points or by any other suitable method or mode.

FIG. 33 illustrates one version of an inflatable device 2602 that is inserted through an access point 2510 in the fractured bone 2500, illustrated in FIG. 22, and into the wedge-shaped cavities 2506, 2508 formed therein. The inflatable device 2602 may have a first wedge-shaped end 2606 and a second wedge shaped end 2608 substantially corresponding to the wedge-shaped cavities 2506, 2508, respectively, in the portions of the fractured bone 2500 and a central longitudinal portion 2613 corresponding to the central bore 2513. The inflatable device 2602 may include a tether 2620 extending therefrom that extends back through an access hole and out of the body for manipulation by a clinician. The tether 2620 may be a tube, a cannulated member, a flexible cord, a rigid rod, a semi-rigid rod, a guide, or any suitable connection and may have a delivery lumen for inflation.

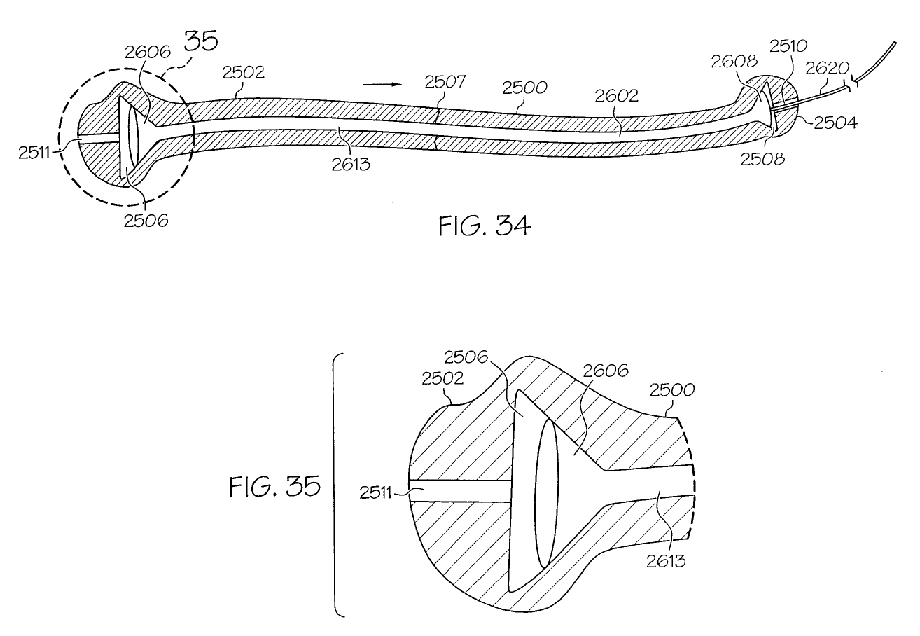

Referring to FIGS. 34-35, the inflatable device 2602 may be inserted into the wedge-shaped cavities 2506, 2508 of the fractured bone 2500 in a deflated state where, upon placement and positioning, the wedge-shaped dumbbell balloon may be inflated. Inflation of the wedge-shaped ends of 2606, 2608 of the inflatable device 2602 may compress the surface of the corresponding wedge-shaped cavities 2506, 2508 such that the expansion of the inflatable device 2602 urges the fractured bone portions 2502, 2504 towards one another. The inflatable device 2602 may also be configured to provide outward resistance or compressive force such that the bone portions 2502, 2504 do not collapse upon each other. Securing the bone portions in both directions may be accomplished, for example, with cavities having a diamond shape corresponding to a balloon having diamond-shaped ends. Still referring to FIGS. 34-35, the wedge-shaped portions or ends 2606, 2608 of the inflatable device 2602 may be inflated until the bone portions 2502, 2504 are rejoined and a desired compressive force is achieved. The compression may be retained, removed, reduced, adjusted, or otherwise manipulated or maintained therapeutically until the bone heals.

Although wedge-shaped cavities and a wedge-shaped dumbbell inflatable device are disclosed by way of example, it will be appreciated that any suitable inflatable device and cavity configuration that, by inflation, rejoins portions of a bone or assists in orthopedic therapy, is contemplated. For example, the inflatable device may have a dumbbell configuration with substantially spherical ends corresponding to spherical cavities. Providing such an inflatable device may beneficially assist in mending bones by providing a compressive force that facilitates bone knitting. Additionally, the central portion of the inflatable device may be inflatable such that an outward pressure is applied to the fractured bone to improve bone stability, shape, and/or healing.

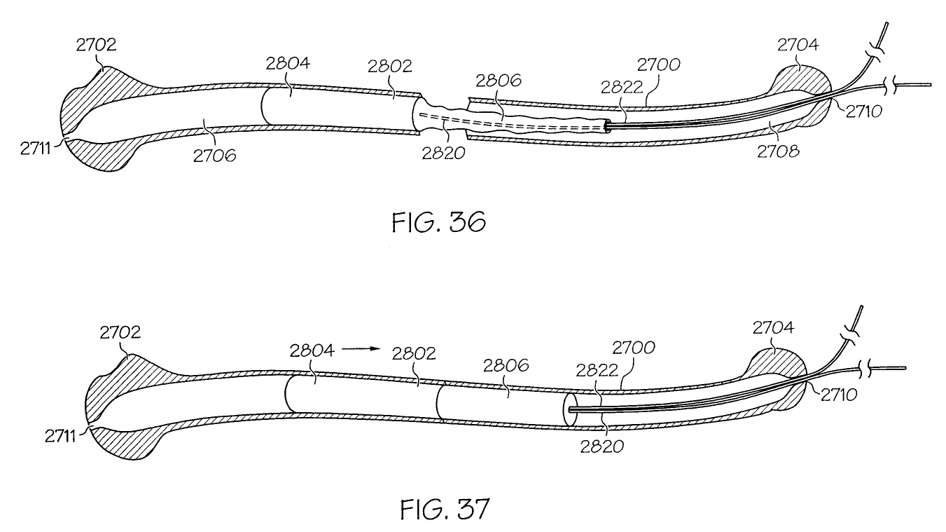

Referring to FIG. 36, one version of an inflatable device 2802 is depicted having a first portion 2804 and a second portion 2806 shown inserted into a fractured bone 2700. The illustrated bone is shown with a first bored or bullet-shaped cavity 2706 and a second bored or bullet shaped cavity 2708 formed in a first bone portion 2702 and a second bone portion 2704, respectively. The cavities 2706, 2708 may be formed in any suitable configuration, such as in a dumbbell shape, a straight bore, or a bullet shape, via access holes 2710, 2711, or the like, and may be created with a bone cutter or any other suitable device. It will be appreciated that the cavities may be formed via one or a plurality of access points or by any other suitable mode.

In one version, the inflatable device 2802 is inserted into an access hole 2710, through the second bone portion 2704, and at least partially into the first bone portion 2702. Once inserted, the first portion 2804 of the inflatable device 2802, which may be a substantially cylindrical balloon portion, may be inflated to engage the inner surface of the first bone portion 2702. The inflation may be accomplished via a first tube 2820, cannulated member, lumen, rod, tether, or the like, where the first tube 2820 passes through the second portion 2806 of the inflatable device 2802 and extends out of the inflatable device 2802 through the access hole 2710. In one version, the first tube is configured to pass around the outer perimeter of the second portion of the inflatable device where, for example, the second portion of the balloon does not have a central bore therethrough. It will be appreciated that any suitable arrangement or configuration of tubes, tethers, inflatable devices, balloons, and/or balloon portions is contemplated. The arrangement may be configured to provide stabilization of the bone segments such that they do not compress and collapse the bone.

Referring to FIG. 37, after inflation of the first portion 2804 of the inflatable device 2802, the first tube 2820 may be pulled or urged proximally in the direction of the access point 2710 such that the first bone portion 2702 engages and/or compresses the second bone portion 2704. After coupling the bone portions 2702, 2704, the second portion 2806 of the inflatable device 2802, which may be a substantially cylindrical balloon portion sharing at least one face with the first portion 2804 of the inflatable device 2802, may be inflated. The second portion 2806 of the inflatable device 2804 may be inflated by a second tube 2822, or the like, such that the inner surface of the fractured bone 2700 is compressed to increase bone stability, or for any other suitable therapeutic benefit.

Providing an inflatable device with at least two chambers, cavities, sections, or the like, may allow various therapeutic functions to be accomplished with a single balloon. For example, as illustrated in FIG. 37, the first portion 2804 of the inflatable device 2802 may be used to grab and axially urge the first bone portion 2702 into contact with the second bone portion 2704. Following this connection or joining of the bone portions 2702, 2704, the second bone portion 2806 may be used to stabilize the bone radially by expanding outward against the inner wall of the bone. Inflating both portions 2804, 2806 of the inflatable device 2802 may allow an axial load and a lateral or radial compressive load to be applied to a bone simultaneously. Such a combination may speed the healing process and may facilitate returning the bone to the original anatomical form. It will be appreciated that any suitable number of chambers having any suitable configuration may be provided in an inflatable device. Multiple chambered inflatable devices with multiple shared or independent lumens are contemplated. A plurality of inflatable devices each having multiple chambers are contemplated. The relationship between inflatable devices, portions of inflatable devices, or combinations thereof, may provide stabilization of the bone segments so that they do not compress and collapse the bone.

It will be appreciated that versions herein have been shown in relation to a long bone, but that versions of the balloon may be configured for any suitable orthopedic application including vertebrae of the spine, the femur, the tibia, the ulna, the radius, or any other suitable bone. While the present invention has been illustrated by description of several embodiments and while the illustrative embodiments have been described in considerable detail, it is not the intention of the applicant to restrict or in any way limit the scope of the appended claims to such detail. Additional advantages and modifications may readily appear to those skilled in the art.

We claim:

1. An inflatable apparatus comprising:
(a) an inflatable member, the inflatable member configured to stabilize a bone, wherein the inflatable member is configured for insertion into a bone cavity; and
(b) a first delivery lumen, wherein the first delivery lumen is coupled with the inflatable member and is configured to inflate the inflatable member.

2. The apparatus of claim 1, further comprising at least one support member associated with the inflatable member.

3. The apparatus of claim 2, wherein the support member is a longitudinally extending member.

4. The apparatus of claim 2, wherein the support member is an annular member.

5. The apparatus of claim 1, wherein the inflatable member includes a first chamber and a second chamber.

6. The apparatus of claim 5, further comprising at least one support member associated with the inflatable member.

7. The apparatus of claim 5, wherein the first chamber and the second chamber are axially aligned.

8. The apparatus of claim 7, wherein the first chamber and the second chamber are longitudinally adjacent.

9. The apparatus of claim 5, wherein the first chamber and the second chamber are coaxial.

10. The apparatus of claim 1, wherein the inflatable member includes projections.

11. The apparatus of claim 10, wherein a first region of projections is provided at a first region of the inflatable member and a second region of projections is provided at a second region of the inflatable member.

12. The apparatus of claim 11, wherein the first region of the inflatable member is at about the proximal end of the inflatable member and the second region of the inflatable member is at about the distal end of the inflatable member.

13. The apparatus of claim 10, wherein the projections are operably configured for inflation.

14. The apparatus of claim 13, wherein the projections are operably configured to be inflated independently from the inflatable member.

15. The apparatus of claim 14, wherein the projections are inflated via a dedicated delivery lumen.

16. The apparatus of claim 10, wherein the projections are fluidly connected with the inflatable member such that inflation of the inflatable member inflates the projections.

17. The apparatus of claim 10, wherein the projections are integral with the inflation member.

18. The apparatus of claim 10, wherein the projections are elements permanently coupled with the inflation member.

19. The apparatus of claim 10, wherein the projections are selected from the group consisting of internal projections, external projections, and combinations thereof.

20. The apparatus of claim 10, wherein the projections are configured for a use selected from the group consisting of increasing the rigidity of the inflatable member, improving the stability of the inflatable member, improving the stability of the bone, reducing the coefficient of friction between the inflatable member and the bone, to prevent fractured portions of the bone from compression upon themselves, and combinations thereof.

21. The apparatus of claim 5, further comprising a second delivery lumen, wherein the first chamber is associated with the first delivery lumen and the second chamber is associated with the second delivery lumen.

22. The apparatus of claim 1, wherein the inflatable member is configured for a use selected from the group consisting of scaffolding, increasing the rigidity of the inflatable member, improving the stability of the inflatable member, improving the stability of the bone, reducing the coefficient of friction between the inflatable member and the bone, to prevent fractured portions of the bone from compression upon themselves, and combinations thereof.

23. A method for stabilizing a bone fracture comprising:
providing a first inflatable apparatus, the first inflatable apparatus comprising;
(a) a first inflatable member, the first inflatable member being configured to stabilize a bone, wherein the first inflatable member is configured for insertion into a first tissue cavity; and
(b) a first tether, wherein the first tether is coupled with the first inflatable member and is configured to inflate the first inflatable member;
providing the first tissue cavity;
inserting the first inflatable apparatus into the first tissue cavity; and
inflating the first inflatable apparatus.

24. The method of claim 23, wherein the first inflatable member is provided with a support member operably configured to stabilize the fractured bone.

25. The method of claim 23, wherein the first inflatable member is provided with a plurality of projections.

26. The method of claim 23, further comprising the steps of;
providing a second inflatable apparatus, the second inflatable apparatus comprising a second inflatable member, the second inflatable member being configured to stabilize the bone, wherein the second inflatable member is configured for insertion into the bone;
inserting the second inflatable apparatus into the bone; and
inflating the second inflatable apparatus.

27. The method of claim 26, wherein the first inflatable member and the second inflatable member are inflated with the first tether.

28. The method of claim 26, wherein the second inflatable apparatus further comprises a second tether, wherein the second tether is coupled with the second inflatable member and is configured to inflate the second inflatable member.

29. The method of claim 28, wherein the first inflatable apparatus and the second inflatable apparatus are coupled.

30. The method of claim 29, wherein the first inflatable apparatus and the second inflatable apparatus are axially coupled.

31. The method of claim 28, further comprising the steps of;
inflating the first inflatable apparatus in the first cavity of the bone; and
inflating the second inflatable apparatus in a second cavity of the bone.

32. The method of claim 31, further comprising the step of joining the bone by manipulating the first inflatable apparatus relative to the second apparatus.

33. The method of claim 32, wherein the step of joining the bone comprises manipulating the first inflatable apparatus with the first tether and the second inflatable apparatus with the second tether.

34. The method of claim 32, wherein the first cavity is in a first bone portion of the fractured bone and the second cavity is in a second bone portion of the bone, wherein the first inflatable apparatus and the second inflatable apparatus are manipulated to join the first bone portion and the second bone portion.

35. The method of claim 32, wherein the first cavity is in a first bone portion and the second cavity is a cavity in the shaft of the bone extending across a fracture in the bone.

36. The method of claim 32, wherein the step of joining the fractured bone further comprises providing an axial force with the first inflatable apparatus and a radial force with the second inflatable apparatus.

37. The method of claim 36, wherein the axial force is provided such that the first bone portion and the second bone portion are restricted from collapsing upon each other.

38. The method of claim 27, further comprising the steps of;
inflating the first inflatable apparatus in the first cavity of the bone; and
inflating the second inflatable apparatus in a second cavity of the bone.
